SEQUENCE LISTING (43 sequences)

SEQ ID NO: 1 - protein, 25 aa, Artificial
  Synthetic peptide
KKAAAXAAAAAXAAXAAXAAAKKKK

SEQ ID NO: 2 - protein, 25 aa, Artificial
  Synthetic peptide
KKAAAXAAAAAXAAWAAXAAAKKKK

SEQ ID NO: 3 - protein, 25 aa, Artificial
  Synthetic peptide
KKAAAFAAAAAFAAWAAFAAAKKKK

SEQ ID NO: 4 - protein, 25 aa, Artificial
  Synthetic peptide
KKAAAWAAAAAWAAWAAWAAAKKKK

SEQ ID NO: 5 - protein, 25 aa, Artificial
  Synthetic peptide
KKAAALAAAAALAAWAALAAAKKKK

SEQ ID NO: 6 - protein, 25 aa, Artificial
  Synthetic peptide
KKAAAIAAAAAIAAWAAIAAAKKKK

SEQ ID NO: 7 - protein, 25 aa, Artificial
  Synthetic peptide
KKAAAYAAAAAYAAWAAYAAAKKKK

SEQ ID NO: 8 - protein, 25 aa, Artificial
  Synthetic peptide
KKAAASAAAAASAAWAASAAAKKKK

SEQ ID NO: 9 - protein, 21 aa, Artificial
  Synthetic peptide
KKAFAAAAAFAAWAAFAKKKK

SEQ ID NO: 10 - protein, 17 aa, Artificial
  Synthetic peptide
KKKKKKAAFAAWAAFAA

SEQ ID NO: 11 - protein, 17 aa, Artificial
  Synthetic peptide
RRRAAFAAWAAFAARRR

SEQ ID NO: 12 - protein, 25 aa, Artificial
  Synthetic peptide
KKAAAAFAAFAAWFAAFAAAAKKKK

SEQ ID NO: 13 - protein, 25 aa, Artificial
  Synthetic peptide
KKAAAMAAAAAMAAWAAMAAAKKKK

SEQ ID NO: 14 - protein, 25 aa, Artificial
  Synthetic peptide
KKAAALAAAAACAAWAALAAAKKKK

SEQ ID NO: 15 - protein, 25 aa, Artificial
  Synthetic peptide
KKATALVGAASLTAWVGLASAKKKK

SEQ ID NO: 16 - protein, 25 aa, Artificial
  Synthetic peptide
KKAAAFAAAAAFAAXAAFAAAKKKK

SEQ ID NO: 17 - protein, 25 aa, Artificial
  Synthetic peptide
KKAAAWAAAAAWAAXAAWAAAKKKK

SEQ ID NO: 18 - protein, 25 aa, Artificial
  Synthetic peptide
KKAAALAAAAALAAXAALAAAKKKK

SEQ ID NO: 19 - protein, 25 aa, Artificial
  Synthetic peptide
KKAAAIAAAAAIAAXAAIAAAKKKK

SEQ ID NO: 20 - protein, 25 aa, Artificial
  Synthetic peptide
KKAAAYAAAAAYAAXAAYAAAKKKK

SEQ ID NO: 21 - protein, 21 aa, Artificial
  Synthetic peptide
KKAFAAAAAFAAXAAFAKKKK

SEQ ID NO: 22 - protein, 16 aa, Artificial
  Synthetic peptide
KKKKKAAAFAAXAAFA

SEQ ID NO: 23 - protein, 17 aa, Artificial
  Synthetic peptide
RRRAAAFAAXAAFARRR

SEQ ID NO: 24 - protein, 25 aa, Artificial
  Synthetic peptide
KKAAAAFAAFAAXFAAFAAAAKKKK

SEQ ID NO: 25 - protein, 25 aa, Artificial
  Synthetic peptide
KKAAAMAAAAAMAAXAAMAAAKKKK

SEQ ID NO: 26 - protein, 25 aa, Artificial
  Synthetic peptide
KKAAALAAAAACAAXAALAAAKKKK

SEQ ID NO: 27 - protein, 25 aa, Artificial
  Synthetic peptide
KKATALVGAASLTAXVGLASAKKKK

SEQ ID NO: 28 - protein, 25 aa, Artificial
  Synthetic peptide
KKAAAVAAAAAVAAWAAVAAAKKKK

SEQ ID NO: 29 - protein, 25 aa, Artificial
  Synthetic peptide
KKAAAAAAAAAAAAWAAAAAAKKKK

SEQ ID NO: 30 - protein, 25 aa, Artificial
  Synthetic peptide
KKAAATAAAAATAAWAATAAAKKKK

SEQ ID NO: 31 - protein, 25 aa, Artificial
  Synthetic peptide
KKAAAGAAAAAGAAWAAGAAAKKKK

SEQ ID NO: 32 - protein, 25 aa, Artificial
  Synthetic peptide
KKAAANAAAAANAAWAANAAAKKKK

SEQ ID NO: 33 - protein, 25 aa, Artificial
  Synthetic peptide
KKKKKKAAAFAAAAAFAAWAAFAAA

SEQ ID NO: 34 - protein, 17 aa, Artificial
  Synthetic peptide
KKKAAAFAAWAAFAKKK

SEQ ID NO: 35 - protein, 17 aa, Artificial
  Synthetic peptide
KKKKKKAAFAAWAAFAA

SEQ ID NO: 36 - protein, 17 aa, Artificial
  Synthetic peptide
RRRRRRAAFAAWAAFAA

SEQ ID NO: 37 - protein, 17 aa, Artificial
  Synthetic peptide
KKKKKKAAAAFWAAAAF

SEQ ID NO: 38 - protein, 17 aa, Artificial
  Synthetic peptide
KKKKKKAAFAAFAAFAA

SEQ ID NO: 39 - protein, 17 aa, Artificial
  Synthetic peptide
KKKKKKAAWAAWAAWAA

SEQ ID NO: 40 - protein, 25 aa, Artificial
  Synthetic peptide
KKAAAVAAAAAVAAXAAVAAAKKKK

SEQ ID NO: 41 - protein, 25 aa, Artificial
  Synthetic peptide
KKAAAAAAAAAAAAXAAAAAAKKKK

SEQ ID NO: 42 - protein, 19 aa, Artificial
  Synthetic peptide
AAAXAAAAAXAAXAAXAAA

SEQ ID NO: 43 - protein, 19 aa, Artificial
  Synthetic peptide
AAAXAAAAAXAAWAAXAAA

(19) United States
(12) Patent Application Publication
(10) Pub. No.: US 2008/0234188 A1
(43) Pub. Date: Sep. 25, 2008

(54) Antimicrobial Peptides

(75) Inventors: Charles M. Deber, Toronto (CA); Li-Ping Liu, Northridge, CA (US)

Correspondence Address: MYERS BIGEL SIBLEY & SAJOVEC, PO BOX 37428, RALEIGH, NC, 27627, US

(73) Assignee: The Hospital for Sick Children

(21) Appl. No.: 11/761,117

(22) Filed: Jun. 11, 2007

Related U.S. Application Data
(63) Continuation of application No. 10/481,286, filed on Jun. 25, 2004, now abandoned.
(60) Provisional application No. 60/299,726, filed on Jun. 22, 2001.

(51) Int. Cl. A61K 38/16; A61K 38/10; C07K 7/08; C07K 14/00; A61P 31/00

(52) U.S. Cl. 514/12; 514/13; 530/324; 530/326

(57) ABSTRACT

A method is described for treating a microbial infection with a peptide whose amino acid sequence has a formula selected from the group consisting of:

(a) Bₙ₁−Z;

(b) Bₙ₁−Z−Bₙ₂; and

(c) Z−Bₙ₁
wherein B is a basic amino acid residue;
n1 and n2 are 1 to 6; and

Z is a sequence of about 11 to about 24 amino acid residues, the sequence having an average hydrophobicity value of at least 0.3, and preferably at least 0.4. These peptides show antimicrobial activity against microorganisms including both Gram-positive and Gram-negative bacteria.

### RELATED APPLICATION INFORMATION

This application is a continuation application of, and claims the benefit under 35 U.S.C. §120 of, U.S. application Ser. No. 10/481,286 filed Jun. 25, 2004, which is a 35 U.S.C. §371 national phase application of PCT Application No. PCT/CA02/00936 filed Jun. 21, 2002, which claims the benefit of U.S. Provisional Application No. 60/299,726 filed Jun. 22, 2001, the disclosures of each of which are incorporated by reference herein in their entireties.

### FIELD OF THE INVENTION

The invention relates to antimicrobial compounds and specifically to novel antimicrobial peptides.

### BACKGROUND OF THE INVENTION

The advent of antibiotics permitted the treatment of bacterial infections and the prevention of many untimely deaths. Widespread use of antibiotics has, however, led to the emergence of antibiotic-resistant strains of many bacteria.

Antibiotic-resistant bacteria can often cause serious illness, and sometimes death, from common bacterial infections in children (Travis (1994), Science, v. 264, pp. 360-362). For example, the deaths of several children between 1997-1999 from infections caused by a resistant strain of methicillin-resistant Staph. aureus were reported by the Center for Disease Control and Prevention in Atlanta. As most well-known antibiotics act by interfering in a specific manner with bacterial homeostasis, bacteria can evolve resistance by mechanisms such as preventing the antibiotic from binding or entering the organism, producing an enzyme that inactivates the antibiotic, and/or changing the internal binding site of the antibiotic. Further examples of antibiotic-resistant bacteria include Enterococcus (vancomycin-resistant); S. pneumoniae (penicillin-resistant); and M. tuberculosis (multi-drug resistant).

New classes of antibiotics must therefore be developed to alleviate the threats to human health arising from antibiotic-resistant bacteria. In this context, antimicrobial peptides offer an attractive alternative. A number of antimicrobial peptides occur naturally as “host-defense” compounds (Oh, J. E. et al., (1999), J. Peptide Res., v. 53, pp. 41-46; Scott, M. G. et al., (1999), Infection & Immunity, v. 67, pp. 2005-2009), in humans (e.g., defensins), other mammals (e.g., bovine granulocyte peptide, described in U.S. Pat. No. 6,008,195), amphibians (e.g., magainins), plants and insects (e.g., cecropins), as well as in bacteria themselves.

Synthetic antimicrobial peptides have also been described, including highly amphipathic peptides whose amino acid sequences are related to or derived from the sequences of various viral membrane proteins, as described in U.S. Pat. No. 5,945,507.

The significant advantage of peptide antimicrobials resides in the global mechanism of their anti-microbial action; because peptides have an inherent capacity to bind and penetrate biological membranes, these compounds act by physically disrupting cellular membranes, usually causing membrane lysis and eventually cell death (LaRocca, P. et al., (1999), Biophys. Chem., v. 76, pp. 145-159). Organisms such as bacteria have little ability to combat this physical mechanism and acquire resistance.

In the past fifteen years, approximately five hundred different antibacterial peptides have been isolated and characterized. They differ widely in length (6-50 residues), sequence and structure, but share two features in that (i) they are generally polycationic; and (ii) their active structures are normally amphipathic, i.e. they usually consist of a mix of positively-charged and non-polar residues alternating in a regular manner along the primary sequence.

Studies of the structure and physical properties of the transmembrane domains of membrane proteins and the structural requirements for the insertion of synthetic peptides into membranes has led to the observation that there is a threshold hydrophobicity requirement for successful peptide insertion into membranes (Liu, L.-P. and Deber, C. (1998), Biopolymers (Peptide Science), v. 47, pp. 41-62; Deber, C. et al., (2001), Protein Science, v.10, pp. 212-219). These studies on laboratory preparations of lipid membranes suggested that the described non-amphipathic peptides could insert into any type of cell membrane, whether mammalian or microbial.

### SUMMARY OF THE INVENTION

The inventors have identified a new group of peptides which have potent antimicrobial activity and no significant cytotoxic effects on eukaryotic cells.

In accordance with one embodiment, the present invention provides a method for treating or preventing a microbial infection in a subject comprising administering to a subject in need of such treatment a peptide in acid or amide form comprising an amino acid sequence having a formula selected from the group consisting of:

(a) Bₙ₁−Z;

(b) Bₙ₁−Z−Bₙ₂; and

(c) Z−Bₙ₁
wherein B is a basic amino acid residue;
n1 and n2 are 1 to 6; and

Z is a sequence of about 11 to about 24 amino acid residues, said sequence having an average hydrophobicity value of at least 0.3, and preferably at least 0.4, in an amount effective to treat or prevent said infection.

In accordance with a further embodiment, the present invention provides a method as described above wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KKAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

In accordance with a further embodiment, the present invention provides a method as described above wherein the peptide is selected from the group consisting of:

| | | |
|---|---|---|
| (a) | KKAAAFAAAAAFAAXAAFAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAFAAAAKKKK-NH₂; | (SEQ ID NO: 24) |
| (j) | KKAAAMAAAAAMAAXAAMAAAKKKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK--NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

In accordance with a further embodiment, the present invention provides a method as described above wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

In accordance with a further embodiment, the present invention provides a method as described above wherein the peptide comprises an amino acid sequence of the formula kkkkkkaafaawaafaa-NH₂ (SEQ ID NO: 35).

In accordance with a further embodiment, the present invention provides a pharmaceutical composition comprising a peptide in acid or amide fomn comprising an amino acid sequence having a formula selected from the group consisting of:

(a) Bₙ₁−Z;

(b) Bₙ₁−Z−Bₙ₂; and

(c) Z−Bₙ₁
wherein B is a basic amino acid residue;
n1 and n2 are 1 to 6; and

Z is a sequence of about 11 to about 24 amino acid residues, said sequence having an average hydrophobicity value of at least 0.3, and preferably at least 0.4, and a pharmaceutically acceptable carrier.

In accordance with a further embodiment, the present invention provides a pharmaceutical composition as described above wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KXAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

In accordance with a further embodiment, the present invention provides a pharmaceutical composition as described above wherein the peptide is selected from the group consisting of:

| | | |
|---|---|---|
| (a) | KKAAAFAAAAAFAAXAAEAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAFAAAAKKKK-NH₂; | (SEQ ID NO: 24) |
| (j) | KKAAAMAAAAAMAAXAAMAAAKXKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK--NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

In accordance with a further embodiment, the present invention provides a pharmaceutical composition as described above wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

In accordance with a further embodiment, the present invention provides use of a peptide in acid or amide form comprising an amino acid sequence having a formula selected from the group consisting of:

(a) Bₙ₁−Z;

(b) Bₙ₁−Z−Bₙ₂; and

(c) Z−Bₙ₁
wherein B is a basic amino acid residue;
n1 and n2 are 1 to 6; and

Z is a sequence of about 11 to about 24 amino acid residues, said sequence having an average hydrophobicity value of at least 0.3, and preferably at least 0.4, to treat or prevent a microbial infection.

In accordance with a further embodiment, the present invention provides use of a peptide in acid or amide form comprising an amino acid sequence having a formula selected from the group consisting of:

(a) Bₙ₁−Z;

(b) Bₙ₁−Z−Bₙ₂; and

(c) Z−Bₙ₁
wherein B is a basic amino acid residue;
n1 and n2 are 1 to 6; and

Z is a sequence of about 11 to about 24 amino acid residues, said sequence having an average hydrophobicity value of at least 0.3, and preferably at least 0.4, in the preparation of a medicament for the treatment or prevention of a microbial infection.

In accordance with a further embodiment, the present invention provides use of a peptide as described above wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KKAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

In accordance with a further embodiment, the present invention provides use of a peptide as described above wherein the peptide is selected from the group consisting of:

| | | |
|---|---|---|
| (a) | KKAAAFAAAAAFAAXAAFAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAFAAAAKKKK-NH₂; | (SEQ ID NO: 24) |
| (j) | KKAAAMAAAAAMAAXAAMAAAKKKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK--NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

In accordance with a further embodiment, the present invention provides use of a peptide as in claim 27 wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

In accordance with a further embodiment, the present invention provides an antimicrobial peptide comprising an amino acid sequence selected from the group consisting of:

| | | |
|---|---|---|
| (a) | KKAFAAAAAFAAWAAFAKKKK-NH₂; | (SEQ ID NO: 9) |
| (b) | KKKKKKAAFAAWAAFAA-NH₂; | (SEQ ID NO: 10) |
| (c) | RRRAAFAAWAAFAARRR-NH₂; | (SEQ ID NO: 11) |
| (d) | KKAAAAFAAFAAWFAAFAAAAKKKK-NH₂; | (SEQ ID NO: 12) |
| (e) | RKATALVGAASLTAWVGLASAKKKK-NH₂. | (SEQ ID NO: 15) |
| (f) | KKKKKKAAAFAAAAAFAAWAAFAAA-NH₂; | (SEQ ID NO: 33) |
| (g) | KKKAAAFAAWAAFAKKK-NH₂; | (SEQ ID NO: 34) |
| (h) | kkkkkkaafaawaafaa-NH₂; | (SEQ ID NO: 35) |
| (i) | RRRRRRAAFAAWAAFAA-NH₂; | (SEQ ID NO: 36) |
| (j) | KKKKKKAAAAFWAAAAF-NH₂; | (SEQ ID NO: 37) |
| (k) | KKKKKKAAFAAFAAFAA-NH₂; | (SEQ ID NO: 38) |
| and | | |
| (l) | KKKKKKAAWAAWAAWAA-NH₂. | (SEQ ID NO: 39) |

### DETAILED DESCRIPTION OF THE INVENTION

The present inventors have identified a new group of peptides which show excellent antimicrobial activity with no significant cytotoxic side effects on eukaryotic cells.

As used herein, an “antimicrobial” peptide is a peptide which inhibits and/or kills pathogenic organisms, for example bacteria, viruses, fungi, yeasts and mycoplasma.

A “microbial infection” is an infection of a subject, or of a tissue or an organ of a subject, by a pathogenic organism, for example a bacterium, a virus, a fungus, a yeast or a mycoplasma. The subject may be a mammal, including a human or a non-human mammal.

“Treating a microbial infection” in a subject means reducing the number of microorganisms infecting the subject and/or reducing the symptoms produced in the subject by the microbial infection.

In contrast to the previously described amphipathic antimicrobial peptides, the peptides of the invention are non-amphipathic, having polar amino acid residues at one or both ends of the peptide but having a non-polar core peptide sequence.

Amino acids are referred to herein in accordance with the standard IUPAC-IUB system of nomenclature, either as one letter or three letter abbreviations.

As used herein: “amino acid” is any amino acid, including the 20 naturally occurring amino acids;

“a basic amino acid” is an amino acid with a basic side chain, for example lysine or arginine;

“hydrophobicity” is a property of an amino acid residue or an amino acid sequence such that the residue or sequence tends to avoid an aqueous environment and tends to locate in a non-polar environment such as the lipid core of a cellular membrane;

“a hydrophobic amino acid” is an amino acid which is not charged at physiological pH and which tends to avoid an aqueous environment and tends to locate in a non-polar environment;

“a hydrophobic amino acid sequence” is an amino acid sequence which contains sufficient hydrophobic amino acids to give the sequence a hydrophobic character.

As used herein, “hydrophobicity” and “hydropathy” are used interchangeably and have the same meaning.

The “hydrophobicity value” of an amino acid residue means the hydrophobicity value of that residue as shown in Table 1 or as calculated by the method described herein.

The “hydrophobicity value” of an amino acid sequence or peptide means the arithmetic average of the individual hydrophobicity values of the constituent amino acid residues of the sequence.

The invention provides methods of treating or preventing microbial infections comprising administering to a subject in need of such treatment an effective amount of a peptide comprising an amino acid sequence having a formula selected from the group consisting of

Bₙ₁−Z, Bₙ₁−Z−Bₙ₂ and Z−Bₙ₁
wherein B is a basic amino acid residue;
n1 and n2 are 1 to 6; and

Z is a sequence of about 11 to about 24 amino acid residues having an average hydrophobicity of at least 0.3.

The invention further provides peptides comprising an amino acid sequence having a formula selected from the group consisting of

Bₙ₁−Z, Bₙ₁−Z−Bₙ₂ and Z−Bₙ₁
wherein B is a basic amino acid residue;
n1 and n2 are 1 to 6; and

Z is a sequence of about 11 to about 24 amino acid residues having an average hydrophobicity of at least 0.3.

In accordance with one embodiment, the peptides used in the method of the invention comprise a hydrophobic “core” amino acid sequence, Z, with at least one basic amino acid residue at each end of the core sequence.

In a further embodiment, the peptides used in the method of the invention comprise a hydrophobic core amino acid sequence with one or more basic amino acid residues at only one end of the core sequence.

The core amino acid sequence, Z, has an average hydrophobicity value of at least 0.3 and preferably at least about 0.4, based on the hydrophobicity scale of Table 1 or by calculation, as described herein.

Peptides as described above which have a core sequence average hydrophobicity of at least 0.3 are active as antimicrobials whereas similar peptides which have a core sequence average hydrophobicity of less than 0.3 are ineffective as antimicrobials (MIC>64 μM).

The peptides of the invention show antimicrobial activity against a broad spectrum of bacteria, including both Gram +ve and Gram −ve bacteria, with MIC values in the low μM range.

The methods of the invention may be employed to treat infections caused, for example, by E. coli, B. subtilis, P. aeruginosa, B. cepacia, S. epidermidis, S. aureus, C. xerosis or E. faecalis.

The methods of the invention may also be used to treat infections caused by yeasts, such as C. albicans, fungi, viruses and mycoplasma.

As will be understood by those of skill in the art, any individual peptide of the invention may show greater antimicrobial activity against some micro-organisms than against others. For example, a couple of the peptides described in Example 5 showed activity against C. xerosis but not against other bacteria tested. It is within the skill of those in the art to screen the peptides of the invention for their level of antimicrobial activity against any particular organism, for example by the tests described in the Examples herein, and to select the peptide or peptides showing the greatest anti-microbial activity against that organism.

Various hydrophobicity scales for proteins and amino acids have been described in the literature but the inventors have established the hydrophobicity scale of Table 1 based on the retention times on HPLC of a series of hydrophobic model peptides, as described in Liu and Deber (1998), Biopolymers (Peptide Science), v. 47, pp. 41-62).

The hydrophobicity value shown for each amino acid in Table 1 as “hydropathy” may be used as the hydrophobicity value for that amino acid in any peptide whose hydrophobicity value has to be calculated. The “average hydrophobicity” of a peptide containing amino acids shown in Table 1 is calculated by determining the arithmetic average of the Table 1 “hydropathy” values for the constituent amino acids of the sequence.

Table 1 lists hydrophobicity or hydropathy values for all the naturally occurring amino acids. Peptides in accordance with the invention may also include other amino acids. For an amino acid not included in Table 1, the hydrophobicity value may be determined by the same method as described herein for obtaining the values of Table 1. For example, a peptide such as KKAAAXAAAAAXAAXAAXAAAKKKK-amide (Sequence ID No: 1) may be synthesized, where X is the amino acid whose hydrophobicity value is to be determined, and the retention time of the peptide on HPLC is determined, as described in the Examples. Similar peptides where X is Lys or Phe are also prepared and their HPLC retention times determined. The retention time, Rx, is converted to the hydropathy value H for the selected amino acid using the equation

H=(10×ΔtR_{x-Lys}/ΔtR_{Phe-Lys})−5.00

where ΔtR_{x-Lys}=retention time difference in minutes between Rₓ and the retention time of the most hydrophilic peptide tested (X=Lys) and
where ΔtR_{Phe-Lys}=retention time difference in minutes between the most hydrophobic peptide tested (X=Phe) and the most hydrophilic (X=Lys).

Within the general formula, B−Z, B−Z−B or Z−B, B may be any basic amino acid, lysine or arginine being preferred.

One or more of the amino acids of the peptides of the invention may be D amino acids. Preferably, one or more of the terminal residues of the core sequence, Z, are D-amino acids.

The C-terminus of the peptides of the invention may be in the free acid form or a pharmaceutically acceptable salt thereof or may be amidated.

In a further embodiment, the core sequence Z is a sequence of hydrophobic amino acids, Hy, with one or more amino acids, X, inserted at any position within the hydrophobic sequence. The hydrophobic amino acids may be exclusively alanine, leucine, valine, isoleucine or phenylalanine or any combination of these amino acids. X may be any amino acid which maintains the average hydrophobicity value of the core sequence at a value of at least 0.3 and preferably at least about 0.4.

In accordance with a further embodiment, the core sequence Z is

Hyₙ₃ X Hyₙ₄ X Hyₙ₅ X Hyₙ₆ X Hyₙ₇

wherein Hy is a hydrophobic amino acid;
X is not present or, if present, is any amino acid; and
n3, n4, n5, n6 and n7 are integers whose total is 10 to 20.

Hy may be the same or different and is preferably an amino acid or amino acids selected from the group consisting of alanine, leucine, valine, isoleucine and phenyl alanine.

X, if present, is preferably selected from the group consisting of alanine, phenylalanine, valine, tryptophan, leucine, isoleucine, methionine, cysteine and tyrosine.

In a preferred embodiment, the peptides of the invention have the general formula KKAAAXAAAAAXAAXAAXAAAKKKK-amide.

Amino acid residues X, interspersed within the alanine chain, may be any amino acid which maintains a core sequence average hydrophobicity value of at least 0.3.

In a further embodiment, the core sequence is Hyₙ₃ X Hyₙ₄ X Hyₙ₅ W Hyₙ₆ X Hyₙ₇ or KKAAAXAAAAAXAAWAAXAAAKKKK-amide (Sequence ID No: 2). The tryptophan residue, W, enables fluorescent detection of the peptides. Hy and X are as defined above. Peptides with core sequence average hydrophobicity values of at least 0.3 were effective antimicrobials.

The peptides of the invention may be synthesized by conventional chemical methods, preferably by solid phase synthesis methods, such as the method described in the Examples herein. Using such methods, D-amino acids may be incorporated into the peptides. Peptides may be purified also by conventional peptide purification methods; exemplary methods are referred to herein.

Alternatively, the peptides of the invention may be made by recombinant expression of nucleotide sequences encoding the desired amino acid sequence, by methods well known to those of skill in the art.

The antimicrobial peptides of the invention may be used to combat a variety of pathogens, including bacteria, viruses, fungi, yeasts and mycoplasma.

The invention provides pharmaceutical compositions comprising at least one peptide of the invention and a pharmaceutically acceptable carrier.

The peptides of the invention may be administered by a variety of routes, including orally, topically, intravenously, subcutaneously, intraocularly, nasally and by inhalation.

The peptides are formulated as required for the particular mode of administration, for example as tablets, pills, powders, capsules and the like, for oral administration, as creams or ointments for topical application or as liquid preparations for intravenous administration.

Suitable formulations and suitable pharmaceutically acceptable carriers for combination with the peptides of the invention as active ingredient are described in standard works such as Remington's Pharmaceutical Sciences, latest edition, Mack Publishing Co., Easton, Pa.

Introduction of D-amino acids into the peptides will assist in protecting the peptides from digestion when administered orally. It may also be desirable to provide the peptides as formulations with an enteric coating for oral administration, such formulations being known to those of skill in the art.

### EXAMPLES

The examples are described for the purposes of illustration and are not intended to limit the scope of the invention.

Methods of chemistry and protein and peptide biochemistry referred to but not explicitly described in this disclosure and examples are reported in the scientific literature and are well known to those skilled in the art.

### Methods

### Materials

Reagents for peptide synthesis, cleavage and purification included Fmoc-protected amino acids (Novabiochem), Fmoc-PAL-PEG-PS resins (Applied Biosystems, CA), N,N-dimethylformamide, peptide grade (Caledon, ON), piperidine (Applied Biosystems, CA, or Acros), methanol (Caledon, ON), N,N-diisopropylethylamine (DIEA) (Aldrich), O-(7-Azabenzotriazol-1-yl)1,1,3,3-tetramethyl-uronium hexaflurophosphate) (HATU) (Applied Biosystems, CA, or GL Biochem Ltd., Shanghai), diethyl ether (Caledon, ON), triisopropylsilane (TIPS) (Aldrich), phenol (Gibco) and acetonitrile (Caledon, ON).

Reagents for micro-BCA protein assay were obtained from Pierce (Rockford, Ill.). Mueller-Hinton broth and Bacto agar were purchased from Difco Laboratories. All other reagents were of analytical grade.

### Bacterial Strains

The bacterial strains used were E. coli C498, E. coli C500, Escherichia coli UB1005 and its antibiotic supersusceptible derivative DC2 (22); a clinical isolate of Staphylococcus epidermidis (C621); coryneform bacterial strain Corynebacterium xerosis (C875); and E. coli ATCC 25922, Staphylococcus aureus ATCC 25923, Enterococcus faecalis ATCC 29212, Bacillus subtilis ATCC 6633 and Pseudomonas aeruginosa ATCC 27853, all from American Type Culture Collection.

### Peptide Synthesis

Peptides were synthesized using standard Fmoc chemistry on a PerSeptive Biosystems Pioneer peptide synthesizer. The synthesis employed the use of the Pioneer's standard (45 min) cycle. A low-load (>0.15 mmol/g) PAL-PEG-PS resin was used to produce an amidated C-terminus. The HATU/DIEA activator pair was used with a 4-fold excess amino acid. Deprotection and cleavage of the peptides were carried out in a mixture of 95% TFA, 2.5% water, 2.5% TIPS (v/v/v) or 88% TFA, 5% phenol, 5% water, 2% TIPS (v/v/v/v), under nitrogen, for 2 hours at room temperature. Cleaved and deprotected peptides were precipitated with ice-cold diethyl ether. Centrifuged pellets were dried, redissolved in water, and lyophilized.

Purification of the peptides was performed on a C4 preparative reverse phase (RP) HPLC column (21.2×250 mm, 300 Å, 10 μm), using a linear gradient of acetonitrile in 0.1% trifluoroacetic acid. Crude peptide (5-12 mg) was dissolved in water and applied to the column. The fraction from the major peak was collected manually, and lyophilized.

Purified peptides were characterized by analytical RP-HPLC, mass spectrometry and amino acid analysis. The RP-HPLC analyses was performed on a Vydac C4 column (4.6×250 mm, 300 Å,5 μm) using a linear gradient of water/0.1% trifluoroacetic acid (A) and acetonitrile/0.1% trifluoroacetic acid (B) at a flow rate of 1 ml/min and 1% B/mm, starting at 10% B. Peptide concentration was determined by amino acid analysis and micro-BCA protein assay.

### Reverse-Phase HPLC

The retention time of each peptide was determined on a C4 reversed-phase column (4.6×250 mm, 300 A pore size, 10 μ particle size). Equal amounts of each peptide were injected into the column and eluted at a flow rate of 1 mL/min, utilizing a linear AB gradient (2% B/min), where buffer A was 0.1% TFA/ddH₂O, and buffer B was 0.1% TFA/acetonitrile. The retention time of each peptide reported here was the average of triplicate measurements.

### Assay of Antibacterial Activity

Peptide antibacterial activity was assessed by the method of R. Hancock et al. (Wu & Hancock (1999), J. Biol. Chem., v. 274, pp. 29-35). Test strains of bacteria from Mueller Hinton agar (MHA) plates were inoculated into 5 ml Mueller Hinton Broth (MHB) in tubes and grown overnight at 37° C. on a shaker (180 rpm). Serial dilutions of test peptides (at 10 times the required test concentrations) were made in 0.01% acetic acid, 0.2% BSA, in polypropylene or coated glass tubes as follows: the test peptide was (a) dissolved in distilled water at 20 times the required maximum concentration (enough final volume for all tests to be performed on a given day); (b) diluted into an equal volume of 0.02% acetic acid, 0.4% BSA to give 10 times the required maximum concentration; (c) serial doubling dilutions were performed in 0.01% acetic acid, 0.2% BSA to provide serial dilutions of peptides at 10 times the required test concentrations, e.g., 640, 320, 160, . . . 2.5 μg/ml. (No deleterious effects of long-term storage of peptides in these media have been detected). Overnight bacterial cultures were diluted in MHB to give 2−7×10⁵ colony forming units/ml. Bacterial suspension (100 μl) was dispensed in each well; controls without bacteria were maintained in parallel. To each well was added 11 μl of 10× test peptide and plates were incubated at 37° C. for 18-24 hours, and checked again at 40-48 hours. Plates were read visually and at 600 nm in a microplate reader (Molecular Devices). Minimum inhibitory concentration (MIC), as used herein, is the lowest concentration of peptide which completely inhibited growth of a tested microorganism.

### Example 1

A number of peptides of the general formula

KKAAXAAAAAXAAWAAXAAAKKKK-amide

were synthesized. The antibacterial activity of these peptides was determined against both Gram-positive bacteria (Streptococcus epidermis, Corynebacterium xerosis) and Gram-negative bacteria (various strains of E. coli). The results are shown in Table 2, along with the average hydrophobicity of each peptide, calculated as described herein.

Peptides of average hydrophobicity of at least 0.3 were antimicrobially active against all bacteria examined (Table 2).

In contrast, no antibacterial activity was detected in peptides with hydrophobicity below the threshold value, even up to 64 μg/ml (S25: Table 2).

The data in Table 2 further show that shortening of the core hydrophobic segment to 15 residues, as in peptide F21; placing six Lys residues at the N-terminus with an F-based core segment of 11 residues [F17(K6)]; and replacing six Lys residues by six arginine (Arg) residues (three at each terminus) (F17R), all result in peptides which retain high antimicrobial activity.

### Example 2

Vero cells (ATCC CCL-81, green monkey kidney) were used to examine any cytotoxic effect of the peptides of the invention on eukaryotic cells, as indicated by their effect on cell growth.

Vero cells were plated to 10,000 cells/well, to give a subconfluent layer. Peptides were added to a final concentration of 320 (μg/ml (peptides dissolved in water at 1.28 mg/ml) and the solution adjusted using 10× PBS solution, to give a saline concentration of (150 mM). 50 μl of this peptide solution was added to wells containing 150 μl of medium. Cell growth was monitored relative to control wells (PBS). Monitoring of cell viability was done using a standard crystal violet assay, in which viable cells are fixed and then stained with crystal violet, solubilized in 10% acetic acid. Absorbances measured at 560 nm are indicative of viable cells. Peptides tested were W25, F25, K25, and S25. The peptides of the invention were not cytotoxic and did not affect cell growth rate (data not shown).

### Example 3

Erythrocytes from heparinized rabbit or human blood were washed three times with phosphate-buffered saline (PBS; 5 mM phosphate buffer, 0.14 M saline, pH 7.3), and centrifuged at 1000×g for 15 min at 4° C. The erythrocytes were diluted with PBS to 4% (v/v). Dilutions of the peptides in PBS were plated in 100 μl volumes in microtitre plates (Costar 3790, polypropylene) together with 100μl of the erythrocyte suspension. PBS was used as control and 100% lysis was determined in 0.1% Triton X-100. The plates were incubated at 37° C. for 1 h and then centrifuged at 1000×g for 5 min. 100 μl aliquots of the supernatant were transferred to microtitre plates (Nunc, polystyrene), and the release of hemoglobin was monitored by measurement of the absorbance at 540 nm in a microplate reader. The results are shown in Table 6, for peptide concentrations of 50 μm and 200 μm. With only one or two exceptions, little or no hemolysis was seen.

### Example 4

Antimicrobial activity of the peptides of the invention was determined for a further range of ATCC strains of pathogenic organisms, as shown below. Peptides tested were F25, F17(K6), and F17R. The set-up for testing the pathogens was generally similar to that described herein for “Assay of Antibacterial Activity”, except that MIC's were tested on sheep blood agar plates (rather than Mueller-Hinton plates); no difference in the amount of colony-forming units was observed between the two plate types.

Antimicrobial activity is indicated as + or ++.

| | |
|---|---|
| Bacillus subtilis | ++ |
| Pseudomonas aeruginosa | + |
| Burkholderia cepacia | + |
| Candida albicans | + |

### Example 5

A further number of the peptides of the invention were tested for antimicrobial activity against a range of Gram-positive and Gram-negative bacteria by the assay described immediately before Example 1.

The results are shown in Tables 3, 4 and 5A and B. Peptide core segment hydrophobicity was calculated as described herein. In a couple of instances, where X=Ala or Val, activity against gram-positive C. xerosis only was seen.

TABLE 1
Hydropathy scale for the 20 commonly-occurring residues determined
from KKAAAXAAAAAXAAWAAXAAAKKKK-amide peptides.

| | | Mean residue hydropathy of |
|---|---|---|
| X-residue | Hydropathy | X-residue peptide |
| Phe | 5.00 | 1.18 |
| Trp | 4.88 | 1.16 |
| Leu | 4.76 | 1.14 |
| Ile | 4.41 | 1.09 |
| Met | 3.23 | 0.90 |
| Val | 3.02 | 0.87 |
| Cys^{a} | 2.49 | 0.78 |
| Tyr | 2.00 | 0.71 |
| Ala | 0.17 | 0.42 |
| Thr | −1.08 | 0.22 |
| Glu | −1.49 | 0.16 |
| Asp | −2.49 | 0.00 |
| Gln | −2.75 | −0.04 |
| Arg | −2.77 | −0.05 |
| Ser | −2.84 | −0.06 |
| Gly | −3.31 | −0.13 |
| Asn | −3.79 | −0.16 |
| His | −4.63 | −0.34 |
| Pro | −4.92 | −0.39 |
| Lys | −5.00 | −0.40 |

^{a}The middle “X” residue was substituted by Cys, and the other two “X” residues were replaced by Leu. Accordingly, the hydropathy of Cys was calculated by the equation: H_{Cys} = [(10 × ΔtR_{Cys-Lys}/ΔtR_{Phe-Lys}) − 5.00 − H_{Leu} × 2/3] × 3.

TABLE 2

| | | | |
|---|---|---|---|
| | | Average | |
| SEQ ID | Peptide | Hydrophobicity | MIC*(μM) |

| | | | | | | | |
|---|---|---|---|---|---|---|---|
| Peptide | No. | Designation | of Core Sequence | C498^{a} | C500^{b} | C621^{c} | C875^{d} |

| | | | | | | | |
|---|---|---|---|---|---|---|---|
| KKAAAFAAAAAFAAWAAFAAAKKKK-NH₂ | 3 | F25 | 1.18 | 8 | 4 | 0.5-2 | 0.25 |
| KKAAAWAAAAAWAAWAAWAAAKKKK-NH₂ | 4 | W25 | 1.16 | 6 | 3 | 0.7 | 0.2 |
| KKAAALAAAAALAAWAALAAAKKKK-NH₂ | 5 | L25 | 1.14 | 6 | 6 | 1.5 | 0.2 |
| KKAAAIAAAAAIAAWAAIAAAKKKK-NH₂ | 6 | I25 | 1.09 | 10 | 10 | 5 | nd |
| KKAAAYAAAAAYAAWAAYAAAKKKK-NH₂ | 7 | Y25 | 0.71 | 11 | 5 | 3 | 0.2 |
| KKAAASAAAAASAAWAASAAAKKKK-NH₂ | 8 | S25 | −0.06 | − | − | − | − |
| KKAFAAAAAFAAWAAFAKKKK-NH₂ | 9 | F21 | 1.45 | 8 | 4 | 8-16 | nd |
| KKKKKKAAFAAWAAFAA-NH₂ | 10 | F17(K6) | 1.48 | 0.5 | <0.25 | nd | 0.5 |
| RRRAAFAAWAAFAARRR-NH₂ | 11 | F17R | 1.48 | 2 | 0.5 | nd | 1 |
| KKAAAAFAAFAAWFAAFAAAAKKKK-NH₂ | 12 | [F(4)25] | 1.43 | 16 | 4 | 8 | 0.25 |
| KKAAAMAAAAAMAAWAAMAAAKKKK-NH₂ | 13 | [M25] | 0.89 | + | + | + | + |
| KKAAALAAAAACAAWAALAAAKKKK-NH₂ | 14 | [C25] | 1.02 | + | + | + | + |
| KKATALVGAASLTAWVGLASAKKKK-NH₂ | 15 | | 0.62 | + | + | + | + |

*MIC = Minimal Inhibitory Concentration
^{a}C498: E. coli, wild type strain
^{b}C500: E. coli, antibiotic strain
^{c}C621: Staphylococcus epidermidis
^{d}C875: Corynebacteriurn xerosis
+ = active, not quantitated
− = inactive
nd, not determined

TABLE 3
MIC's of the 25 residue KKAAAXAAAAAXAAWAAXAAAKKKK-amide
peptides against Gram-negative and Gram-positive bacteria

| | | | MIC (μM)^{a} | | | |
|---|---|---|---|---|---|---|
| | | | Gram-negative | | Gram-positive | |
| | | Mean | bacteria | | bacteria | |
| Peptide | Sequence | Core segment | E. coli | E. coli | C. xerosis | S. epidermidis |
| X residue | ID No. | Hydrophobicity | DC2 | UB1005 | C875 | C621 |
| F | 3 | 1.18 | 4 | 16 | <0.25 | 2 |
| W | 4 | 1.16 | 8 | 16 | <0.25 | <0.25 |
| L | 5 | 1.14 | 16 | 32 | <0.25 | 2 |
| I | 6 | 1.09 | 16 | >32 | <0.25 | 4 |
| M | 13 | 0.90 | 32 | >32 | 0.5 | 16 |
| V | 28 | 0.87 | >32 | >32 | 0.5 | >32 |
| C | 14 | 1.02 | 8 | 16 | 1 | >32 |
| Y | 7 | 0.71 | 16 | 32 | <0.25 | 8 |
| A | 29 | 0.42 | >64 | >64 | 2 | >64 |
| T | 30 | 0.22 | >64 | >64 | >64 | >64 |
| S | 8 | −0.06 | >64 | >64 | >64 | >64 |
| G | 31 | −0.13 | >64 | >64 | >64 | >64 |
| N | 32 | −0.21 | >64 | >64 | >64 | >64 |

^{a}Values are representative of three or more separate experiments.

TABLE 4

Amino acid sequences and hydrophobicity of
antimicrobial peptides.

| | | | |
|---|---|---|---|
| | SEQ | | Mean Core |
| | ID | | segment hy- |
| Peptide | No. | Amino acid sequence | drophobicity |

| | | | |
|---|---|---|---|
| F25 | 3 | KKAAAFAAAAAFAAWAAFAAAKKK | 1.18 |
| | | K-NH₂ | |
| F25-6K | 33 | KKKKKKAAAFAAAAAFAAWAAFAA | 1.18 |
| | | A-NH₂ | |
| 4F | 12 | KKAAAAFAAFAAWFAAFAAAAKKK | 1.43 |
| | | K-NH₂ | |
| F21 | 9 | KKAFAAAAAFAAWAAFAKKKK-NH₂ | 1.45 |
| F17 | 34 | KKKAAAFAAWAAFAKKK-NH₂ | 1.47 |
| F17-R | 11 | RRRAAFAAWAAFAARRR-NH₂ | 1.47 |
| F17-6K | 10 | KKKKKKAAFAAWAAFAA-NH₂ | 1.47 |
| A11-D- | 35 | kkkkkkaafaawaafaa-NH₂ | 1.47 |
| F17-6K | | | |
| F17-6R | 36 | RRRRRRAAFAAWAAFAA-NH₂ | 1.47 |
| KAFW | 37 | KKKKKKAAAAFWAAAAF-NH₂ | 1.47 |
| 3F17-6K | 38 | KKKKKKAAFAAFAAFAA-NH₂ | 1.49 |
| W17-6K | 39 | KKKKKKAAWAAWAAWAA-NH₂ | 1.45 |

^{a}For clarity, the Phe and Trp residues are in bold. Amino acids in lower case are D-enantiomers.

TABLE 5A
MIC's of peptides against Gram-negative bacteria.

| | | MIC (μM)^{a} | | | |
|---|---|---|---|---|---|
| | SEQ | Gram-negative bacteria | | | |
| | ID | E. coli | E. coli | E. coli | P. aeruginosa |
| Peptide | No. | DC2 | UB1005 | ATCC25922 | ATCC27853 |
| F25 | 3 | 4 | 16 | >32 | 8 |
| F25-6K | 33 | 8 | 16 | 16 | 32 |
| 4F | 12 | n.d. | 8 | 16 | 8 |
| F21 | 9 | 4 | 8 | 32 | >32 |
| F17 | 34 | 16 | 32 | >32 | >32 |
| F17-R | 11 | 1 | 2 | 8 | 16 |
| F17-6K | 10 | 0.5 | 1 | 8 | 16 |
| A11-D F17-6K | 35 | n.d. | 0.5 | 2 | 8 |
| F17-6R | 36 | 0.5 | 1 | 4 | 8 |
| KAFW | 37 | 0.5 | 2 | 16 | 16 |
| 3F17-6K | 38 | n.d. | 2 | 16 | 16 |
| W17-6K | 39 | n.d. | 1 | 8 | 8 |

n.d., not determined
^{a}Values are representative of three or more separate experiments.

TABLE 5B
MIC's of peptides against Gram-positive bacteria.

| | | MIC (μM)^{a} | | | | |
|---|---|---|---|---|---|---|
| | | Gram-positive bacteria | | | | |
| | SEQ ID | C. xerosis | S. epidermidis | S. aureus | E. faecalis | B. subtilis |
| Peptide | No. | C875 | C621 | ATCC25923 | ATCC29212 | ATCC6633 |
| F25 | 3 | <0.25 | 2 | >32 | >32 | 1 |
| F25-6K | 33 | 1 | 4 | n.d | n.d. | n.d. |
| 4F | 12 | n.d. | 4 | 32 | >32 | 2 |
| F21 | 9 | 0.5 | 8 | >32 | >32 | 2 |
| F17 | 34 | 4 | >32 | >32 | >32 | 16 |
| F17-R | 11 | 1 | 4 | >32 | >32 | 2 |
| F17-6K | 10 | 0.5 | 4 | >32 | >32 | 8 |
| A11-D F17-6K | 35 | n.d. | 4 | 32 | >32 | 4 |
| F17-6R | 36 | 0.5 | 4 | n.d. | n.d. | n.d. |
| KAFW | 37 | 0.5 | 8 | n.d. | n.d. | n.d. |
| 3F17-6K | 38 | n.d. | 8 | >32 | >32 | 16 |
| W17-6K | 39 | n.d. | 8 | 32 | >32 | 4 |

n.d., not determined
^{a}Values are representative of three or more separate experiments.

TABLE 6
Hemolytic activity of antimicrobial peptides in rabbit human
erythrocytes (red blood cells, RBC)

| | |
|---|---|
| Rabbit RBC | Human RBC |
| (% lysis) | (% lysis) |

| Peptide | 200 μM | 50 μM | 200 μM | 50 μM |
|---|---|---|---|---|
| S25 | 3 | 0 | 0 | 0 |
| A25 | 1 | 0 | n.d. | n.d. |
| F25 | 4 | 0 | 1 | 0 |
| F25-6K | n.d. | n.d. | 17 | 11 |
| 4F | 34 | 14 | 42 | 17 |
| F21 | 3 | 1 | 0 | 0 |
| F17 | 2 | 0 | 0 | 0 |
| F17-R | 2 | 0 | 0 | 0 |
| F17-6K | 1 | 0 | 0 | 0 |
| A11-D F17-6K | 2 | 0 | 0 | 0 |
| F17-6R | 3 | 1 | 26 | 14 |
| KAFW | 1 | 0 | 0 | 0 |
| 3F17-6K | 2 | 0 | 0 | 0 |
| W17-6K | 2 | 0 | 0 | 0 |

n.d. not determined
Percentages are given to nearest +/−0.5%.
Values are representative of 2-3 experiments.

1. A method for treating or preventing a microbial infection in a subject comprising administering to a subject in need of such treatment a peptide in acid or amide form comprising an amino acid sequence having a formula selected from the group consisting of:
(a) Bₙ₁- Z;
(b) Bₙ₁-Z-Bₙ₂; and
(c) Z-Bₙ₁
wherein B is a basic amino acid residue;
n1 and n2 are 1 to 6; and
Z is a sequence of about 11 to about 24 amino acid residues, said sequence having an average hydrophobicity value of at least 0.3, and preferably at least 0.4, in an amount effective to treat or prevent said infection.

2. The method of claim 1 wherein Z is a sequence of about 14 to about 20 amino acids.

3. The method of claim 1 or 2 wherein Z contains one tryptophan residue.

4. The method of claim 1 wherein each B is independently lysine or arginine; and
Z is a sequence of 19 amino acids, each amino acid being selected independently from the group consisting of alanine, leucine, valine, isoleucine, phenylalanine, tryptophan, methionine, tyrosine and cysteine.

5. The method of claim 1 wherein each B is independently lysine or arginine; and
Z is a sequence of about 10 to about 23 hydrophobic amino acids having inserted singly at any position within said sequence from 1 to 4 further amino acids, X, where X is any amino acid.

6. The method of claim 1 wherein B is lysine; and
Z is Hyₙ₃ X Hyₙ₄ X Hyₙ₅ W Hyₙ₆ X Hyₙ₇
wherein Hy is a hydrophobic amino acid;
X is not present or, if present, is any amino acid; and
n3, n4, n5, n6 and n7 are integers whose total is 10 to 20.

7. The method of claim 5 or 6 wherein X is selected from the group consisting of alanine, phenylalanine, tryptophan, leucine, isoleucine, methionine, cysteine and tyrosine.

8. The method of claim 1 wherein the peptide comprises an amino sequence having the formula:
KKAAAXAAAAAXAAXAAXAAAKKKK-amide wherein X is any amino acid.

9. The method of claim 8 wherein one X residue of said peptide is replaced by W.

10. The method of claim 1 wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KKAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

11. The method of claim 1 wherein the peptide is selected from the group consisting of:

| | | |
|---|---|---|
| (a) | KKAAAFAAAAAFAAXAAFAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAEAAAAKKKK-NH₂; | (SEQ ID NO: 24) |
| (j) | KKAAAMAAAAAMAAXAAMAAAKKKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK--NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

12. The method of claim 1 wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

13. The method of any one of claims 1 to 12 wherein at least one amino acid of said peptide is a D-amino acid.

14. The method of claim 13 wherein the peptide comprises an amino acid sequence of the formula kkkkkkaafaawaafaa-NH₂ (SEQ ID NO: 35).

15. The method of any one of claims 1 to 14 wherein the microbial infection is a bacterial infection.

16. The method of claim 15 wherein the bacterial infection is a Gram-positive bacterial infection.

17. The method of claim 15 wherein the bacterial infection is a Gram-negative bacterial infection.

18. The method of claim 15 wherein the bacterial infection is an infection by a bacterium selected from the group consisting of E. coli, B. subtilis, F. aeruginosa, B. cepacia, S. epidermidis, S. aureus, C. xerosis and F. faecalis.

19. The method of any one of claims 1 to 14 wherein the microbial infection is a fungal or yeast infection.

20. The method of claim 19 wherein the infection is a C. albicans infection.

21. The method of any one of claims 1 to 20 wherein the peptide is administered by a route of administration selected from the group consisting of oral, topical, intravenous, subcutaneous, nasal and by inhalation.

22. A pharmaceutical composition comprising a peptide in acid or amide form comprising an amino acid sequence having a formula selected from the group consisting of:
(a) Bₙ₁-Z;
(b) Bₙ₁-Z-Bₙ₂; and
(c) Z-Bₙ₁
wherein B is a basic amino acid residue;
n1 and n2 are 1 to 6; and
Z is a sequence of about 11 to about 24 amino acid residues, said sequence having an average hydrophobicity value of at least 0.3, and preferably at least 0.4, and a pharmaceutically acceptable carrier.

23. The pharmaceutical composition of claim 22 wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KKAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

24. The pharmaceutical composition of claim 22 wherein the peptide is selected from the group consisting of:

| | | |
|---|---|---|
| (a) | KKAAAFAAAAAFAAXAAFAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAFAAAAKKKK-NH₂; | (SEQ ID NO: 24) |
| (j) | KKAAAMAAAAAMAAXAAMAAAKKKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK--NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

25. The pharmaceutical composition of claim 22 wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

26. Use of a peptide in acid or amide form comprising an amino acid sequence having a formula selected from the group consisting of:
(a) Bₙ₁-Z;
(b) Bₙ₁-Z-Bₙ₂; and
(c) Z-Bₙ₁
wherein B is a basic amino acid residue;
n1 and n2 are 1 to 6; and
Z is a sequence of about 11 to about 24 amino acid residues, said sequence having an average hydrophobicity value of at least 0.3, and preferably at least 0.4, to treat or prevent a microbial infection.

27. Use of a peptide in acid or amide form comprising an amino acid sequence having a formula selected from the group consisting of:
(a) Bₙ₁-Z;
(b) Bₙ₁-Z-Bₙ₂; and
(c) Z-Bₙ₁
wherein B is a basic amino acid residue;
n1 and n2 are 1 to 6; and
Z is a sequence of about 11 to about 24 amino acid residues, said sequence having an average hydrophobicity value of at least 0.3, and preferably at least 0.4, in the preparation of a medicament for the treatment or prevention of a microbial infection.

28. Use of a peptide as in claim 27 wherein Z is a sequence of about 14 to about 20 amino acids.

29. Use of a peptide as in claim 27 or 28 wherein Z contains one tryptophan residue.

30. Use of a peptide as in claim 27 wherein each B is independently lysine or arginine; and
Z is a sequence of 19 amino acids, each amino acid being selected independently from the group consisting of alanine, leucine, valine, isoleucine, phenylalanine, tryptophan, methionine, tyrosine and cysteine.

31. Use of a peptide as in claim 27 wherein each B is independently lysine or arginine; and
Z is a sequence of about 10 to about 23 hydrophobic amino acids having inserted singly at any position within said sequence from 1 to 4 further amino acids, X, where X is any amino acid.

32. Use of a peptide as in claim 27 wherein B is lysine; and
Z is Hyₙ₃ X Hyₙ₄ X Hyₙ₅ W Hy_{n6 X} Hyₙ₇
wherein Hy is a hydrophobic amino acid;
X is not present or, if present, is any amino acid; and
n3, n4, n5, n6 and n7 are integers whose total is 10 to 20.

33. Use of a peptide as in claim 31 or 32 wherein X is selected from the group consisting of alanine, phenylalanine, tryptophan, leucine, isoleucine, methionine, cysteine and tyrosine.

34. Use of a peptide as in claim 27 wherein the peptide comprises an amino sequence having the formula:
KKAAAXAAAAAXAAXAAXAAAKKKK-amide wherein X is any amino acid.

35. Use of a peptide as in claim 34 wherein one X residue of said peptide is replaced by W.

36. Use of a peptide as in claim 27 wherein the peptide is selected from the group consisting of:

| | | |
|---|---|---|
| (a) | KKAAAFAAAAAFAAWAAFAAAKKKK-NH₂; | (SEQ ID NO: 3) |
| (b) | KKAAAWAAAAAWAAWAAWAAAKKKK-NH₂; | (SEQ ID NO: 4) |
| (c) | KKAAALAAAAALAAWAALAAAKKKK-NH₂; | (SEQ ID NO: 5) |
| (d) | KKAAAIAAAAAIAAWAAIAAAKKKK-NH₂; | (SEQ ID NO: 6) |
| (e) | KKAAAYAAAAAYAAWAAYAAAKKKK-NH₂; | (SEQ ID NO: 7) |
| (f) | KKAFAAAAAFAAWAAFAKKKKK-NH₂; | (SEQ ID NO: 9) |
| (g) | KKKKKKAAFAAWAAFAA-NH₂; | (SEQ ID NO: 10) |
| (h) | RRRAAFAAWAAFAARRR-NH₂; | (SEQ ID NO: 11) |
| (i) | KKAAAAFAAFAAWFAAFAAAAKKKK-NH₂; | (SEQ ID NO: 12) |
| (j) | KKAAAMAAAAAMAAWAAMAAAKKKK-NH₂; | (SEQ ID NO: 13) |
| (k) | KKAAALAAAAACAAWAALAAAKKKK-NH₂; | (SEQ ID NO: 14) |
| (l) | KKATALVGAASLTAWVGLASAKKKK-NH₂. | (SEQ ID NO: 15) |
| (m) | KKAAAVAAAAAVAAWAAVAAAKKKK--NH₂; | (SEQ ID NO: 28) |
| and | | |
| (n) | KKAAAAAAAAAAAAWAAAAAAKKKK--NH₂. | (SEQ ID NO: 29) |

37. Use of a peptide as in claim 27 wherein the peptide is selected from the group consisting of:

| | | |
|---|---|---|
| (a) | KKAAAFAAAAAFAAXAAFAAAKKKK-NH₂; | (SEQ ID NO: 16) |
| (b) | KKAAAWAAAAAWAAXAAWAAAKKKK-NH₂; | (SEQ ID NO: 17) |
| (c) | KKAAALAAAAALAAXAALAAAKKKK-NH₂; | (SEQ ID NO: 18) |
| (d) | KKAAAIAAAAAIAAXAAIAAAKKKK-NH₂; | (SEQ ID NO: 19) |
| (e) | KKAAAYAAAAAYAAXAAYAAAKKKK-NH₂; | (SEQ ID NO: 20) |
| (f) | KKAFAAAAAFAAXAAFAKKKK-NH₂; | (SEQ ID NO: 21) |
| (g) | KKKKKAAAFAAXAAFA-NH₂; | (SEQ ID NO: 22) |
| (h) | RRRAAAFAAXAAFARRR-NH₂; | (SEQ ID NO: 23) |
| (i) | KKAAAAFAAFAAXFAAFAAAAKKKK-NH₂; | (SEQ ID NO: 24) |
| (j) | KKAAAMAAAAAMAAXAAMAAAKKKK-NH₂; | (SEQ ID NO: 25) |
| (k) | KKAAALAAAAACAAXAALAAAKKKK-NH₂; | (SEQ ID NO: 26) |
| (l) | KKATALVGAASLTAXVGLASAKKKK-NH₂; | (SEQ ID NO: 27) |
| (m) | KKAAAVAAAAAVAAXAAVAAAKKKK-NH₂; | (SEQ ID NO: 42) |
| and | | |
| (n) | KKAAAAAAAAAAAAXAAAAAAKKKK--NH₂. | (SEQ ID NO: 43) |

wherein X is any hydrophobic amino acid of hydropathy value greater than or equal to alanine.

38. Use of a peptide as in claim 27 wherein the peptide is selected from the group consisting of:

| | | |
|---|---|---|
| (a) | KKKKKKAAAFAAAAAFAAWAAFAAA-NH₂; | (SEQ ID NO: 33) |
| (b) | KKKAAAFAAWAAFAKKK-NH₂; | (SEQ ID NO: 34) |
| (c) | RRRRRRAAFAAWAAFAA-NH₂; | (SEQ ID NO: 36) |
| (d) | KKKKKKAAAAFWAAAAF-NH₂; | (SEQ ID NO: 37) |
| (e) | KKKKKKAAFAAFAAFAA-NH₂; | (SEQ ID NO: 38) |
| and | | |
| (f) | KKKKKKAAWAAWAAWAA-NH₂. | (SEQ ID NO: 39) |

39. Use of a peptide as in any one of claims 27 to 38 wherein at least one amino acid of said peptide is a D-amino acid.

40. Use of a peptide as in claim 39 wherein the peptide comprises an amino acid sequence of the formula kkkkkkaafaawaafaa-NH₂ (SEQ ID NO: 35).

41. Use of a peptide as in any one of claims 27 to 40 wherein the microbial infection is a bacterial infection.

42. Use of a peptide as in claim 41 wherein the bacterial infection is a Gram-positive bacterial infection.

43. Use of a peptide as in claim 41 wherein the bacterial infection is a Gram-negative bacterial infection.

44. Use of a peptide as in claim 41 wherein the bacterial infection is an infection by a bacterium selected from the group consisting of E. coli, B. subtilis, P. aeruginosa, B. cepacia, S. epidermidis, S. aureus, C. xerosis and F. faecalis.

45. Use of a peptide as in any one of claims 27 to 40 wherein the microbial infection is a fungal or yeast infection.

46. Use of a peptide as in claim 45 wherein the infection is a C. albicans infection.

47. Use of a peptide as in any one of claims 27 to 46 wherein the peptide is administered by a route of administration selected from the group consisting of oral, topical, intravenous, subcutaneous, nasal and by inhalation.

48. An antimicrobial peptide comprising an amino acid sequence selected from the group consisting of:

| | | |
|---|---|---|
| (a) | KKAFAAAAAFAAWAAFAKKKK-NH₂; | (SEQ ID NO: 9) |
| (b) | KKKKKKAAFAAWAAFAA-NH₂; | (SEQ ID NO: 10) |
| (c) | RRRAAFAAWAAFAARRR-NH₂; | (SEQ ID NO: 11) |
| (d) | KKAAAAFAAFAAWFAAFAAAAKKKK-NH₂; | (SEQ ID NO: 12) |
| (e) | KKATALVGAASLTAWVGLASAKKKK-NH₂. | (SEQ ID NO: 15) |
| (f) | KKKKKKAAAFAAAAAFAAWAAFAAA-NH₂; | (SEQ ID NO: 33) |
| (g) | KKKAAAFAAWAAFAKKK-NH₂; | (SEQ ID NO: 34) |
| (h) | kkkkkkaafaawaafaa-NH₂; | (SEQ ID NO: 35) |
| (i) | RRRRRRAAFAAWAAFAA-NH₂; | (SEQ ID NO: 36) |
| (j) | KKKKKKAAAAFWAAAAF-NH₂; | (SEQ ID NO: 37) |
| (k) | KKKKKKAAFAAFAAFAA-NH₂; | (SEQ ID NO: 38) |
| and | | |
| (l) | KKKKKKAAWAAWAAWAA-NH₂. | (SEQ ID NO: 39) |
